(19) United States
(12) Patent Application Publication
(10) Pub. No.: US 2025/0279157 A1
(43) Pub. Date: Sep. 4, 2025

(54) A METHOD AND SYSTEM FOR FAST END-TO-END LEARNING ON PROTEIN SURFACES

(71) Applicants: ÉCOLE POLYTECHNQUE FÉDÉRALE DE LAUSANNE (EPFL), Lausanne (CH); IMPERIAL COLLEGE INNOVATIONS LIMITED, London (GB)

(72) Inventors: Michael Bronstein, London (GB); Freyr Sverrisson, St. Sulpice (CH); Jean Bao Pierre Feydy, London (GB); Pablo Gainza, Lausanne (CH); Bruno Emanuel Ferreira De Sousa Correia, St-Saphorin- sur-Morges (CH)

(21) Appl. No.: 18/266,293

(22) PCT Filed: Dec. 10, 2021
(86) PCT No.: PCT/EP2021/085326

§ 371 Date: Jun. 9, 2023

Related U.S. Application Data
(60) Provisional application No. 63/124,217, filed on Dec. 11, 2020.

(51) Int. Cl. G16B 15/30; G16B 15/20; G16B 40/20

(52) U.S. Cl. CPC G16B 15/30; G16B 15/20; G16B 40/20

(57) ABSTRACT

The present invention concerns a computer-system-implemented method for predicting properties of a protein molecule, comprising the steps of: receiving an input representation of the protein molecule; applying a surface generator to produce a molecular surface; applying at least one layer of geometric convolution on the molecular surface to produce a set of surface features; and using the set of features to predict the properties of the molecule.

### CROSS REFERENCE TO RELATED APPLICATIONS

This patent application is a national stage application, filed under 35 U.S.C. § 371, of International Application No. PCT/EP2021/085326, filed Dec. 10, 2021 and entitled “A Method And System For Fast End-To-End Learning On Protein Surfaces”. In addition, this application claims the benefit of U.S. Provisional Application No. 63/124,217 (filed on Dec. 11, 2020).

### BACKGROUND

Proteins' biological functions are defined by the geometric and chemical structure of their 3D molecular surfaces. Recent works have shown that geometric deep learning can be used on mesh-based representations of proteins to identify potential functional sites, such as binding targets for potential drugs. Unfortunately though, the use of meshes as the underlying representation for protein structure has multiple drawbacks including the need to pre-compute the input features and mesh connectivities. This becomes a bottleneck for many important tasks in protein science.

### 1. INTRODUCTION

Proteins are biomacromolecules central to all living organisms. Their function is a determining factor in health and disease, and being able to predict functional properties of proteins is of the utmost importance to developing novel drug therapies. From a chemical perspective, proteins are polymers composed of a sequence of amino acids, see e.g., FIG. 1A. This sequence determines the structural conformation (fold) of the protein, and the structure in turn determines its function. In a folded protein, hydrophobic (water-repelling) residues typically cluster within the core of the protein, while hydrophilic (water-attracting) residues are exposed to water solvent on its surface. The properties of this surface dictate the type and the strength of the interactions that a protein can have with other molecules, see e.g., FIG. 1B. Analysing this complex 3D object is therefore a fundamental problem in biology: models for protein structures can be used to understand the possible interactions between a protein and its environment, and consequently predict the functions of these macromolecules in living organisms.

FIGS. 1A and 1B demonstrate three major problems in structural biology. As shown in FIG. 1A, protein design is the inverse problem of structure prediction. FIG. 1B shows two interacting proteins represented as an atomic point cloud (left) and as a molecular surface (right) that abstracts out the internal fold (shown semi-transparently). Protein surfaces display a number of geometric (e.g. concave and convex regions) and chemical (e.g. charges) features. Identifying the binding of the protein surfaces is a complex problem that can be addressed with geometric deep learning.

Since proteins are predominant drug targets, the study of their interactions with other molecules is a key problem for fundamental biology and the pharmaceutical industry. Classical drugs are small molecules designed to bind to a protein of interest, with a binding site that usually has noticeable ‘pocket-like’ structure. Targets with flat surfaces that exhibit no pockets have long been a challenge for drug developers and are often deemed ‘undruggable’. The possibility of addressing such targets with specifically designed protein molecules (known as biological drugs or ‘biologics’) is a fast emerging field in drug-development holding the promise to provide novel therapeutic strategies for many important diseases (e.g. cancer, viral infections).

Deep learning methods have increasingly been applied to a broad range of problems in protein science, with the particularly notable success of DeepMind's AlphaFold to predict 3D protein structure from sequence. Recently, the Molecular Surface Interaction Fingerprinting (MaSIF) method was introduced by Pablo Gainza, Freyr Sverrisson, Frederico Monti, Emanuele Rodola, D Boscaini, M M Bronstein, and B E Correia introduced in their paper Deciphering interaction fingerprints from protein molecular surfaces using geometric deep learning as published in Nature Methods, 17(2):184-192, 2020 (herein after Gainza 2020), which is incorporated by reference herein in its entirety. MaSIF is one of the first conceptual approaches for geometric deep learning on protein molecular surfaces allowing prediction of their binding. The main limitations of MaSIF stem from its reliance on precomputed meshes and handcrafted features, as well as significant computational time and memory requirements.

### 2. RELATED WORKS

### Deep Learning in Protein Science

Proteins can be represented in different ways, the 1D amino acid sequence being the simplest and most abundant source of data. Recent methods have taken advantage of the wealth of protein sequences available in public databases and shown how unsupervised embeddings borrowed from the field of Natural Language Processing can improve function prediction. Deep learning is also becoming a key component in many pipelines for protein folding (i.e. inferring the 3D structure from the amino acid sequence). These methods often predict pair-wise distances and other geometric relations between different residues to use them as constraints in later structural refinements. Relations between amino acids of different proteins have also been predicted to handle protein-protein interactions. Protein design can be considered as ‘inverse structure prediction’ (i.e. predict a sequence that will fold into a particular structure) and has also benefited from deep learning methods.

Surface representations are relevant to the field: they abstract the internal parts of the protein fold which do not contribute to interactions. The MaSIF method pioneered the use of mesh-based geometric deep learning to predict protein interactions. It was used to classify binding sites for small ligands, discriminate sites of protein-protein interaction in surfaces and predict protein-protein complexes.

Nevertheless, in spite of its conceptual importance and impressive performance, the MaSIF method has significant drawbacks that limit its practical applications for protein prediction and design. First, it takes as inputs mesh-based representations of a protein surface, that must be generated from the raw atomic point cloud as a preprocessing step. Second, it relies on hand-crafted chemical and geometric features that must also be pre-computed and stored on the hard drive. Third, it uses MoNet mesh convolutions on precomputed geodesic patches, which becomes prohibitively expensive in terms of memory and run time when working with more than a few thousand proteins.

### Deep Learning on Surfaces and Point Clouds

Deep learning on non-Euclidean structured data such as meshes, graphs, and point clouds, known under the umbrella term geometric deep learning, has recently become an important tool in computer vision and graphics. Instead of considering geometric shapes as objects in a 3D Euclidean space and applying standard deep learning pipelines (e.g. based on 2D views, volumetric, space partitioning, and implicit representations, geometric deep learning seeks to develop a non-Euclidean analogy of filtering and pooling operations. The first geometric convolutional neural network like (CNN-like) architecture (Geodesic CNN) was based on intrinsic local charting on meshes. Follow up works improved on these results using patch operators based on anisotropic diffusion (ACNN), Gaussian mixtures (MoNet, splines, graph message passing (FeastNet), equivariant filters, and primal-dual mesh operators.

Point clouds are often used as a native representation of 3D data coming from range sensors, and have recently gained popularity in computer vision in lieu of surface based representations. First works on deep learning on point clouds were based on deep learning on sets (PointNet and PointNet++). Dynamic Graph CNN (DGCNN) uses graph neural networks on k-nearest neighbors (kNN) graphs constructed on the fly to capture the local structure of the point cloud. Additional tangent space and volumetric convolution operators were also considered.

### SUMMARY

Described herein is a new framework for deep learning on protein structures that addresses limitations with use of meshes as the underlying representation for protein structure. Among the key advantages of the method described herein are the computation and sampling of the molecular surface on-the-fly from the underlying atomic point cloud and a novel efficient geometric convolutional layer. As a result, large collections of proteins in an end-to-end fashion are able to be processed while taking as the sole input the raw 3D coordinates and chemical types of the protein's atoms, eliminating the need for any hand-crafted pre-computed features.

To showcase the performance of the approach described herein, testing on two tasks in the field of protein structural bioinformatics were conducted: the identification of interaction sites and the prediction of protein-protein interactions. On both tasks, state-of-the-art performance was achieved with much faster run times and fewer parameters than previous models. These results will considerably case the deployment of deep learning methods in protein science and open the door for end-to-end differentiable approaches in protein modeling tasks such as function prediction and design.

### Main Contributions

Described herein is a differentiable molecular surface interaction fingerprinting (dMaSIF) deep learning approach to identifying interaction patterns on protein surfaces that addresses the key drawbacks of MaSIF. The dMaSIF architecture/method is free of any precomputed features, operates directly on the large set of atoms that compose the protein, generates a point cloud representation for the protein surface, learns task-specific geometric and chemical features on the surface point cloud, and finally applies a new convolutional operator that approximates geodesic coordinates in the tangent space. All these computations are performed on the fly, with a small memory footprint. Notably, all core calculations are implemented as reductions of “symbolic matrices”, supported by the recent KeOps library for PyTorch. These high performance routines enable design of a method which is fully differentiable and an order of magnitude faster and more memory efficient than MaSIF. This in turn allows predictions to be made on larger collections of protein structures than was previously practical, and opens the door to end-to-end protein optimization and de nova protein design using geometric deep learning.

### BRIEF DESCRIPTION OF THE DRAWINGS

FIG. 1A illustrates the relationship between protein design and structure prediction;

FIG. 1B shows an atomic point cloud and molecular surface representation of two interfacing proteins;

FIG. 2 shows a flow chart of methods for successive geometric representation of proteins;

FIGS. 3A-3F show a sampling method for protein surfaces;

FIGS. 4A-4G illustrate the binding of a 10J7 protein pair;

FIG. 5A-5C illustrate an implementation of fast quasi-geodesic convolutions on oriented point clouds as an approximation of the geodesic distance;

FIGS. 6A and 6B show experimental results from computing chemical properties of a protein surface from an underlying atomic point using a network according to disclosed embodiments;

FIG. 7 is a graph of ROC curves comparing performance of the dMaSIF method of disclosed embodiment to the prior art MaSIF method;

FIG. 8 is a graph showing accuracy vs run time for different mode architectures;

FIG. 9 is a graph showing accuracy vs memory footprint for different mode architectures;

FIG. 10 shows a schematic diagram of a network architecture for site identification according to disclosed embodiments;

FIG. 11 shows a schematic diagram of a network architecture for interaction prediction according to disclosed embodiments;

FIG. 12 shows a schematic diagram of a network architecture for constructing a surface representation according to disclosed embodiments;

FIG. 13 shows a schematic diagram of a network architecture for estimating chemical features from raw atom types and coordinates according to disclosed embodiments;

FIG. 14 shows a schematic diagram of a convolutional network architecture according to disclosed embodiments;

FIGS. 15A and 15B are graphs demonstrating quality control for surface generation methods according to disclosed embodiments;

FIGS. 16A-16C are graphs illustrating computational cost as a function of batch size for different processes of the disclosed embodiments;

FIGS. 17A-17C are graphs illustrating computational cost as a function of resolution for different processes of the disclosed embodiments;

FIG. 18A is a rendering of a ground truth electrostatic potential for a 10J7_D protein;

FIG. 18B is a rendering of an electrostatic potential for a 10J7_D protein predicted by processes of the disclosed embodiments;

FIG. 18C is a rendering of the error between the ground truth of FIG. 18A and the prediction of FIG. 18B; and

FIG. 19 is a graphical display output of a site prediction task of the disclosed embodiments.

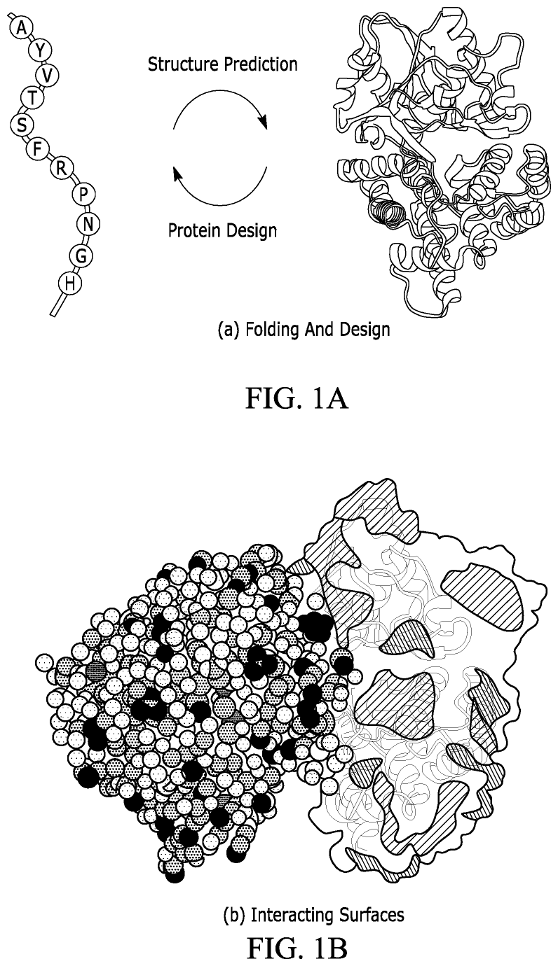

### DETAILED DESCRIPTION

### 3. APPROACH

### Working With Protein Surfaces

In the following, a new efficient end-to-end architecture for geometric deep learning on protein molecules is described. The premise described herein is that protein molecular surfaces carry important geometric and chemical information that is indicative of the way the protein molecular surfaces interact with other molecules. Though the method is showcased on predicting binding properties (arguably, the most important task in structural biology and drug design), it is generic and can be trained on other problems—and in principle, be extended to other biomolecules.

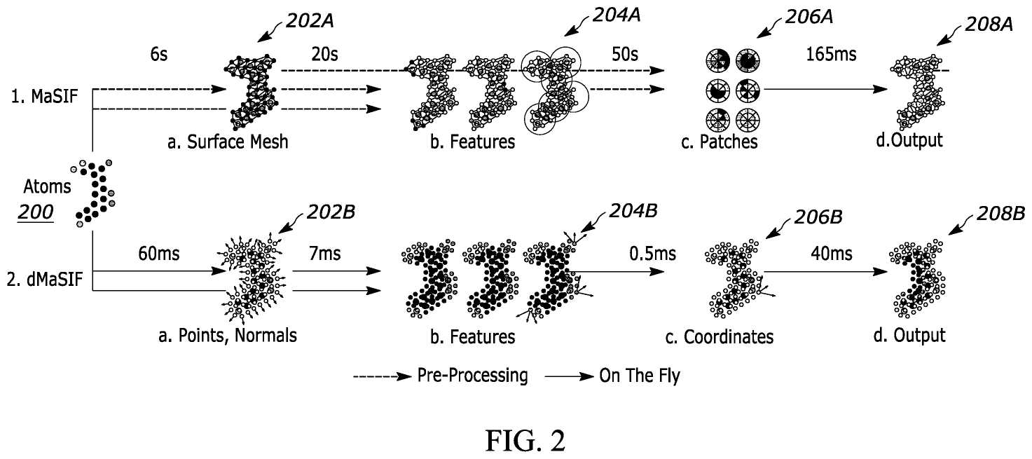

The method described herein works on successive geometric representations of a protein as illustrated in FIG. 2. In particular, FIG. 2 shows that both MaSIF and dMaSIF go through the same steps for interface prediction on protein surfaces. Starting from a raw atomic point cloud 200, representations 202A and 202B of the protein molecular surface are calculated at step (a), geometric and chemical features 204A and 204B are calculated at steps (b), and local coordinate systems 206A and 206B are calculated at steps (c). Then, binding sites 208 are predicted by a geometric convolutional neural network operating on (quasi-) geodesic patches on the protein surface at steps (d). The MaSIF process precomputes steps (a), (b), and (c) shown in FIG. 2, whereas the dMaSIF process computes steps (a), (b), and (c) shown in FIG. 2 on the fly 600 times faster than with the MaSIF proc. For every step, FIG. 2 shows an average run time per protein for inference on the site prediction task as described in Section 4 below. The dMaSIF method results in an accuracy level on par with MaSIF while alleviating the need for pre-calculations and providing significant speed-up for both inference and training.

Furthermore, the input to the MaSIF and dMaSIF processes is provided as a cloud of atoms 200 {a₁, . . . , a_{A}}⊂R³ with chemical types in the list [C, H, O, N, S, Se] encoded as one-hot vectors {t₁, . . . , t_{A}}⊂R⁶. The surface of the protein is then represented as an oriented point cloud {x₁, . . . , x_{N}}⊂R³ with unit normals \hat{n}₁, . . . , \hat{n}_{N} in R³. Feature vectors f₁, . . . , f_{N} are associated to the points in the point cloud and progressively updates using convolution-like operations; the dimension of these features varies from 16 (10 geometric+6 chemical features as input) to 1 (binding score as output) throughout the network. The data comes from the Protein Data Bank, with protein structures that are typically made up of A=3K-15K atoms and molecule sizes in the range 30 Å-300 Å (one ångström is equal to 10⁻¹⁰ m); the surfaces are sampled at a resolution of 1 Å to work with N=6K-15K points at a time.

Unlike most other works for surface processing, the method described herein does not rely on mesh structures, kNN graphs, or space partitioning of any kind. Exact interactions between all points of a protein surface are computed efficiently using the recent KeOps library for PyTorch that optimizes a wide range of computations on generalized distance matrices. The size 5K-20K and dimension 3 of the point clouds appear to be a sweetspot for KeOps in ‘bruteforce mode’, thanks to contiguous operations that stream much better on GPUs than the scattered memory accesses of graph-based and hierarchical methods.

### 3.1. Surface Generation

### Fast Sampling

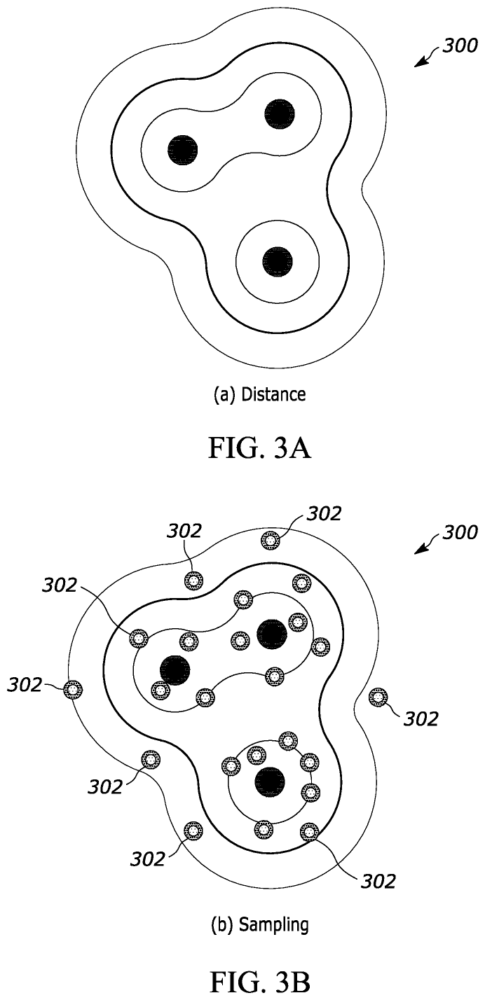

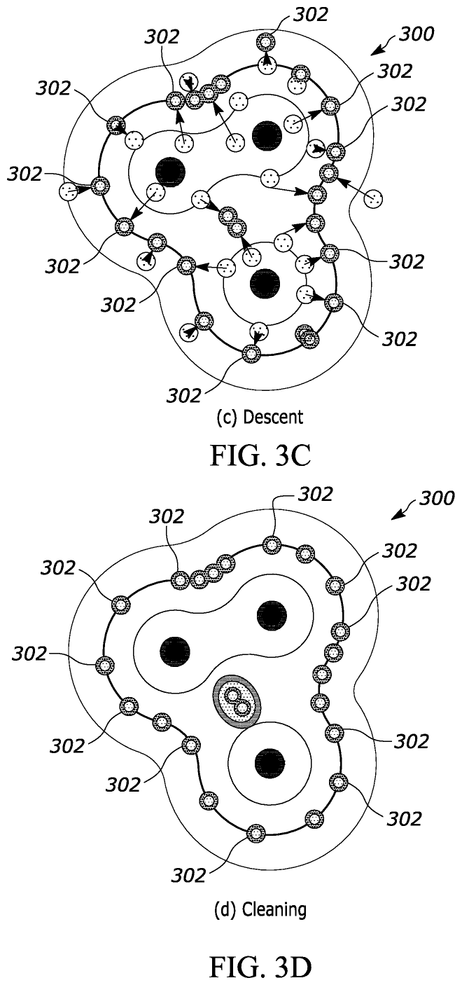

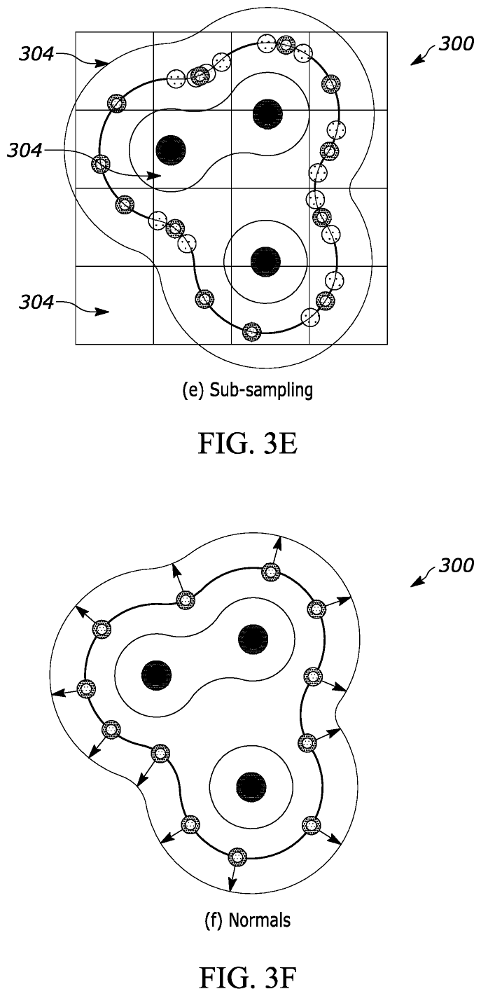

The surface of a protein can he described as the level set of a smooth distance function or meta ball. For example, see FIG. 3A showing a given input protein 300 encoded as an atomic point cloud a₁, . . . , a_{A} with its molecular surface represented as a level set of the smooth distance function (1) to the atom centers. To represent the six different atom types accurately, we associate an atomic radius σₖ to each atom aₖ and define the smooth distance function:

$$\text{?}$$

$$\text{?} \text{indicates text missing or illegible when filed}$$

for any x∈R³, with a stable log-sum-exp reduction and with:

$$\text{?}$$

$$\text{?} \text{indicates text missing or illegible when filed}$$

the average atom radius in a neighborhood of point x.

As shown in FIG. 3B, the level set surface is sampled at radius r=1.05 Å by minimizing the squared loss function:

$$\text{?}$$

$$\text{?} \text{indicates text missing or illegible when filed}$$

on a random Gaussian sample. KeOps allows implementation of this sampling strategy efficiently on batches of more than 100 proteins at a time.

To sample the surface shown in FIG. 3B, a point cloud 302 of x₁, . . . , x_{N=AB} is generated in the neighborhood of the protein 300. For every atom center, B=20 points from N(μ=aₖ, σ=10 Å) are drawn and, as shown in FIG. 3C, this random sample is converged towards the target level set by gradient descent on (2)-4 gradient steps with a learning rate of 1 are used. Then, as shown in FIG. 3D, any points trapped inside the protein 300 are removed. A sample is kept if the distance function at this location is close to the target value of r=1.05 Å within a margin of 0.10 Å, and if making four consecutive steps of size 1 Å in the direction of the gradient of the distance function increases it by more than 0.5 Å. Then, as shown in FIG. 3E, all points are put in cubic bins 304 of side length 1 Å and keep one average sample per cell. This process ensures that the sampling has uniform density. Finally, as shown in FIG. 3F, the gradient of the distance function at location xᵢ is normalized to be used as a normal \hat{n}ᵢ.

### Descriptors

Point normals \hat{n}ᵢ are computed using the gradient of the distance function (1). To estimate a local coordinate system (\hat{n}ᵢ, ûᵢ, \hat{v}ᵢ), vector field is first smoothed using a Gaussian kernel with aσ∈{9,12}Å, i.e. use \hat{n}ᵢ←Normalize (Σⱼ₌₁^{N}exp(−∥xᵢ−xⱼ∥²/2σ²)\hat{n}ⱼ). Tangent vectors ûᵢ and \hat{v}ᵢ are computed using the efficient formulae described in Building an orthonormal basis, revisited. JCGT, 6(1), 2017. Let \hat{n}ᵢ=[x,y,z] be a unit vector, s=sign(z), a=−1/(s+z) and b=a×y, then:

$${\hat{u}}_{i} = [ 1 + {sax}^{2} , sb , - sx ] , {\hat{v}}_{i} = [ b , s + {ay}^{2} , - y ] . \qquad ( 3 )$$

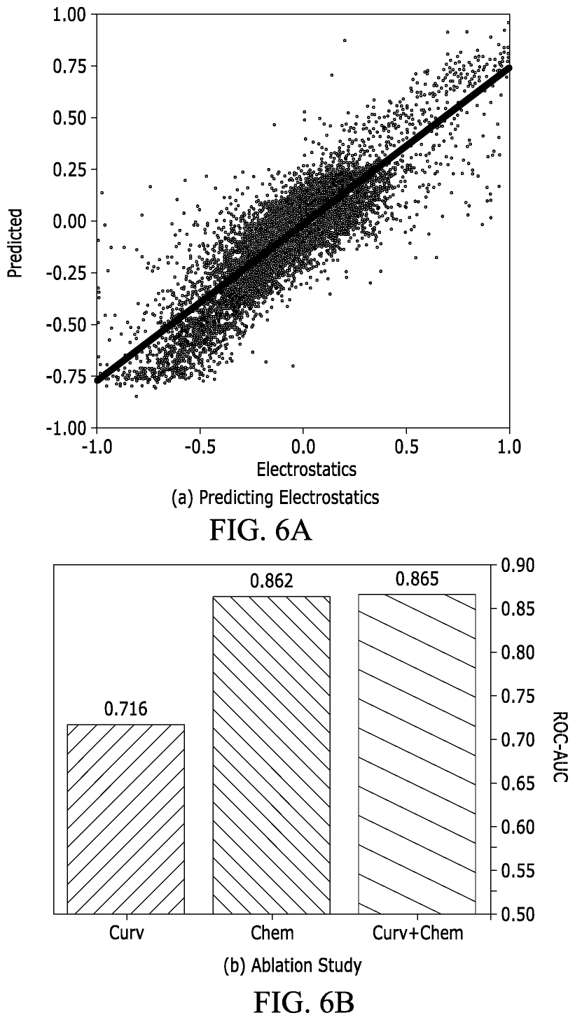

For each point xᵢ, we then find the 16 nearest atom centers {a₁ⁱ, . . . , a₁₆ⁱ} with types {t₁ⁱ,. . . , t₁₆ⁱ} encoded as onehot vectors in R.6. We compute a vector of chemical features fi in R⁶ by applying a Multi-Layer Perceptron (MLP) to the vectors [tⁱₖ, 1/∥xᵢ−aⁱₖ∥] in R⁷, performing a summation over the indices k=1, . . . , 16 and applying a second MLP to the result. As illustrated in FIG. 6 and described in more detail below, using simple MLPs with a single hidden layer of dimension 12 is enough to learn rich chemical features, such as the Poisson-Boltzmann electrostatic potential.

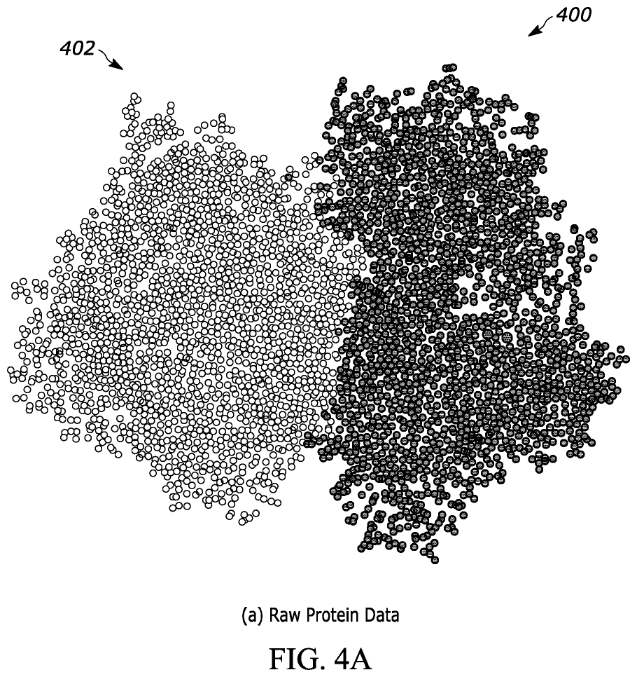

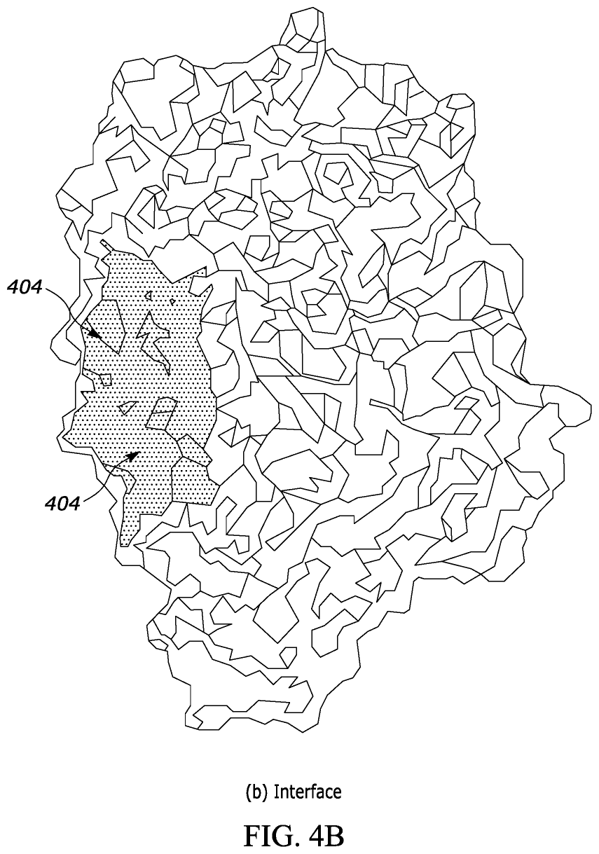

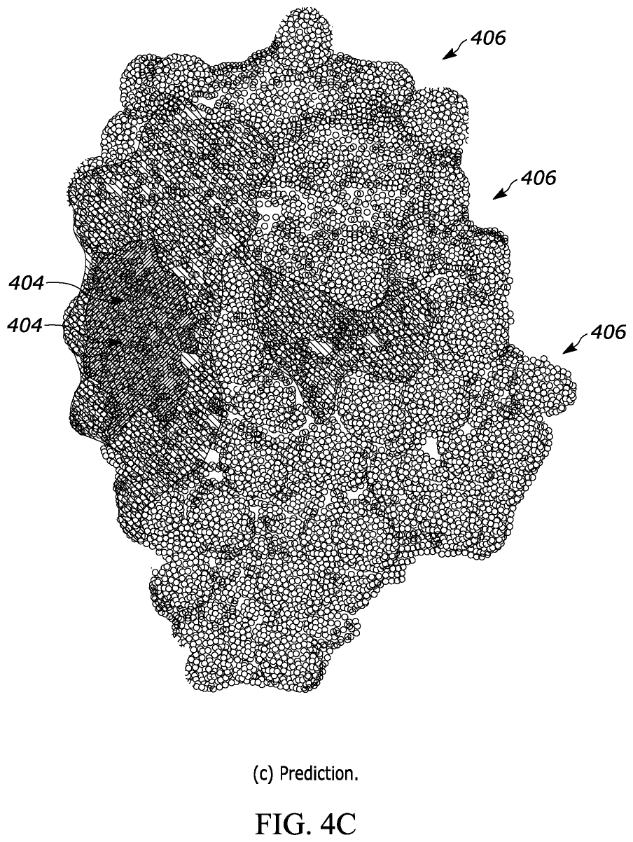

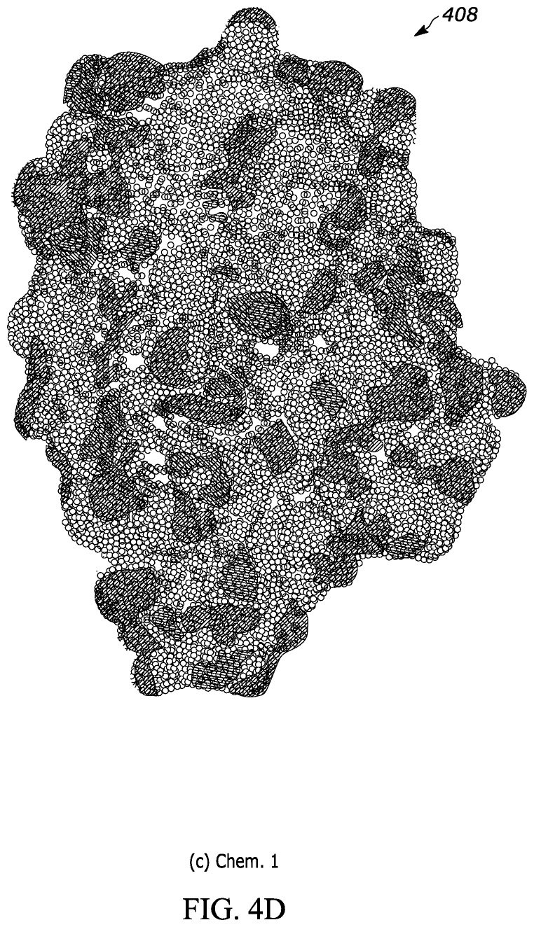

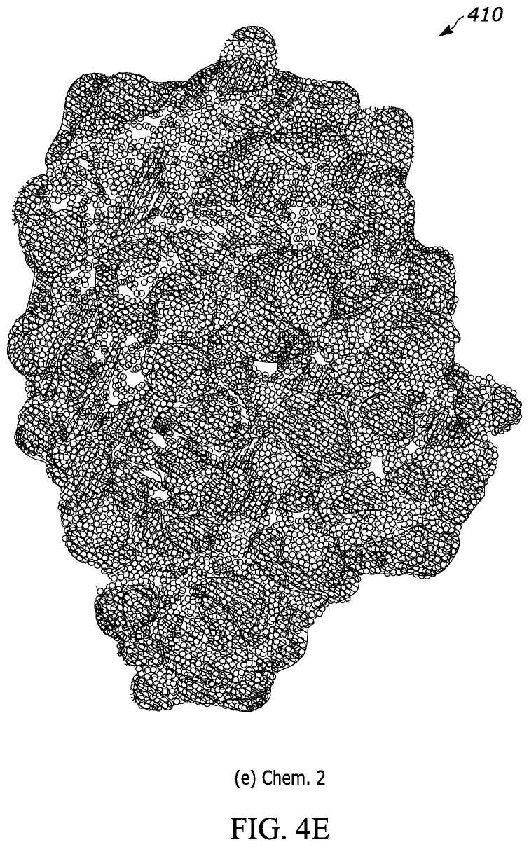

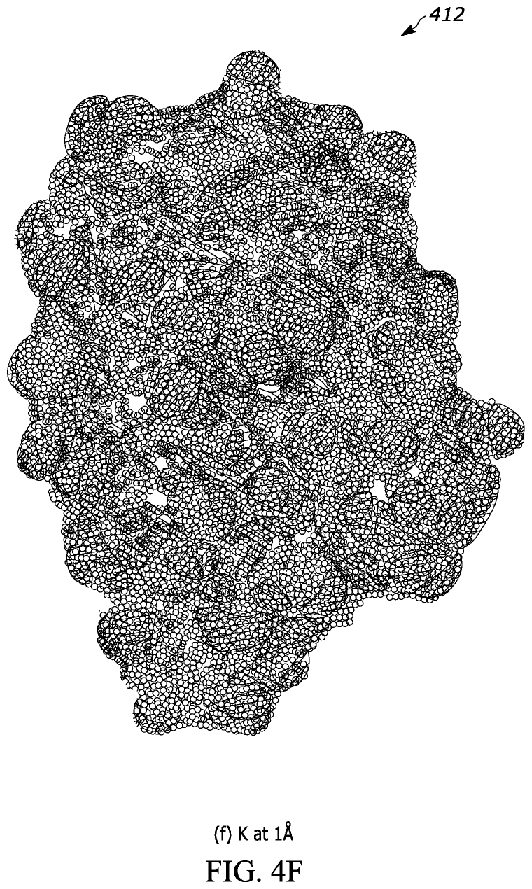

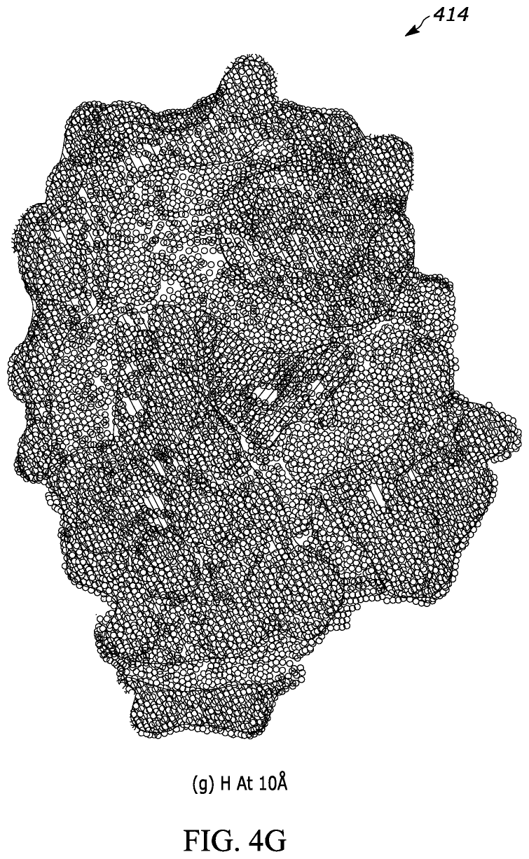

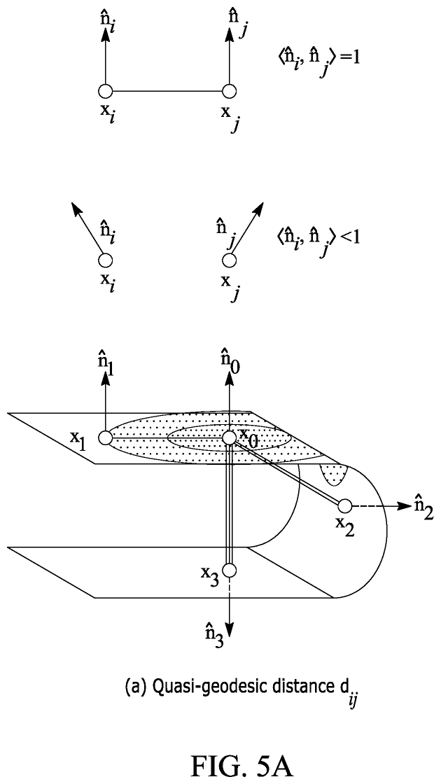

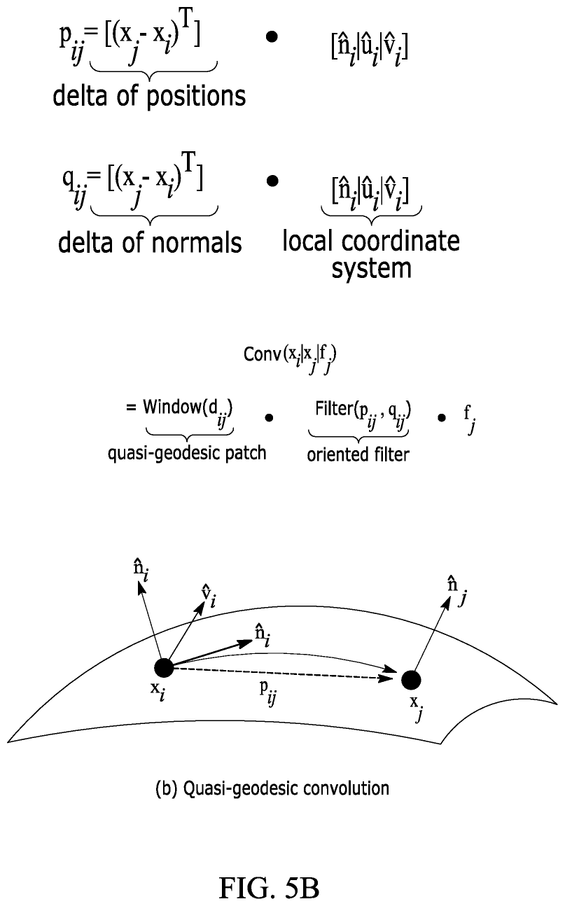

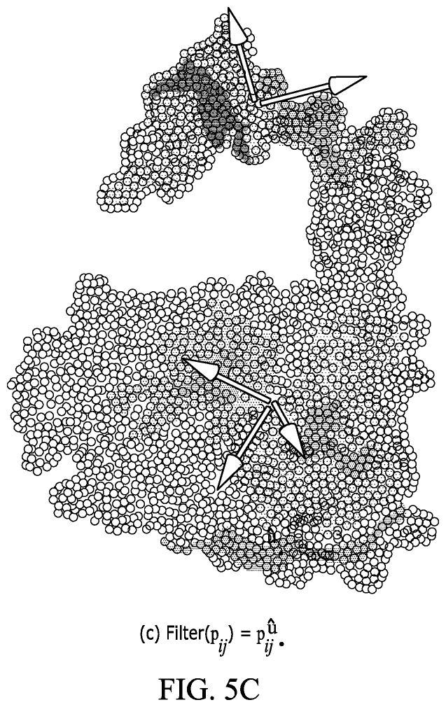

FIGS. 4A-4G illustrate and example binding of the 10J7 protein pair. In particular, FIG. 4A show proteins 10J7_D 400 and 10J7_A 402 for which the Protein Data Bank documents interactions the between. The ability to predict this 3D binding configuration can be learned from the unregistered structures of both proteins. MaSIF tackles this problem as a surface segmentation problem. For example, as shown in FIG. 4B a binding site 404 is the ground truth signal that MaSIF tries to predict from precomputed chemical and geometric features, such as the electrostatic potential. MaSIF relies on mesh convolutions on the preprocessed molecular surface of the protein. The dMaSIF method predicts the binding site 404 without using any precomputed mesh structure or features. Instead, the computations are performed on an oriented point cloud 406, as shown in FIG. 4C, that is generated from the raw atom coordinates as described in connection with FIGS. 3A-3F. Data-driven chemical features 408 and 410 shown in FIGS. 3D and 3E as well as Gaussian curvatures 412 and mean curvatures 414 shown in FIGS. 3F and 3G, respectively, at different scales are computed on the fly and given as inputs to a fast convolutional architecture described in connection with FIGS. 5A-5C.

### 3.2. Quasi-Geodesic Convolutions on Point Clouds

### Convolutions on 3D Shapes

To update the feature vectors fᵢ and progressively learn to predict the binding site of a protein, (quasi-)geodesic convolutions on the molecular surface are relied on. This ensures that the model is fully invariant to 3D rotations and translations, takes decisions according to local chemical and geometric properties of the surface, and is not influenced by atoms located deep inside the volume of a protein. These modelling hypotheses hold for many protein interaction problems and prevent the network from overfitting on the few thousands of protein pairs that are present in the dataset.

In practice, geometric convolutional networks combine pointwise operations of the form fᵢ′←MLP(fᵢ) with local inter-point interactions of the form:

$$\text{?}$$

$$\text{?} \text{indicates text missing or illegible when filed}$$

where fᵢ and \acute{f}ᵢ denote feature vectors associated to the point xᵢ and the Conv(xᵢ, xⱼ, fⱼ) operator puts a trainable weight on the relationship between the points xᵢ and xⱼ. The sum can possibly be replaced by a maximum or any other reduction or pooling operation.

### Working With Oriented Point Clouds

Numerous methods have been proposed to mimic surface operators with convolution operators on meshes or point clouds—see Section 2. The systems and method described here leverage the normal vectors that are produced by the sampling algorithm to define a fast quasi-geodesic convolutional layer that works directly on oriented point clouds. The KeOps library lets this operation be implemented efficiently, without any offline precomputation on the surface geometry.

As illustrated in FIG. 5, the geodesic distance between two points xᵢ and xⱼ of a protein surface with unit normals \hat{n}ᵢ and \hat{n}ⱼ is approximated as:

$${d}_{ij} = ‖ {x}_{i} - {x}_{j} ‖ \cdot ( 2 〈 {\hat{n}}_{i} , 〉 \qquad ( 5 )$$

and filters are localized using a smooth Gaussian window of radius σ∈{9,12}Å, w(dᵢⱼ)=exp(−d²ᵢⱼ/2σ²). In the neighborhood of any point xⱼ of the surface, two 3D vectors then encode the relative position and orientation of neighbor points xj in the local coordinate system (\hat{n}ᵢ, ûᵢ, \hat{v}ᵢ):Pᵢⱼ=[Pᵢⱼ^{\hat{n}}, Pᵢⱼ^{û}, Pᵢⱼ^{\hat{v}}], Qᵢⱼ=[Qᵢⱼ^{\hat{n}}, Qᵢⱼ^{û}, Qᵢⱼ^{\hat{v}}]. Different choices for the trainable “Filter” on these 3D vectors let a wide range of operations to be encoded. The focus here is on polynomial functions and MLPs instead of the popular Mixture-of-Gaussian filters, but note that this choice has little impact on the expressive power of the model.

In more detail, FIG. 5A shows that the weighted distance dᵢⱼ between points xᵢ and xⱼ is equal to ∥xᵢ-xⱼ∥ if the unit normal vectors \hat{n}ᵢ and \hat{n}ⱼ point towards the same direction, but is larger otherwise. In the example of FIG. 5A, the points x₁, x₂ and x₃ lay at equal distance of the reference point x₀ in R³; but since the reference normal \hat{n}₀ is aligned with \hat{n}₁, orthogonal to \hat{n}₂ and opposite to \hat{n}₃ we have d_{0,1}=∥x₀-x₁∥<2·d_{0,1}=d_{0,2}<3·d_{0,1}=d_{0,3}. FIG. 5B shows leveraging of this behavior to prevent information leakage “across the volume” of a protein. A Gaussian window on the weighted distance dᵢⱼ is combined with a parametric “Filter” to aggregate features fⱼ between neighbors on a protein surface. FIG. 5C shows a local coordinate systems induced by the formulae. This local coordinate system closely mimics the structure of genuine geodesic patches—defined here by a Gaussian window of deviation σ=10 Å. On smooth surfaces, they enable the computation of “quasi-geodesic” convolutions at a much lower cost than mesh-based methods.

### Local Orientation, Curvatures

It should be stressed, however, that the pair of tangent vectors (ûᵢ, \hat{v}ᵢ), orthogonal to the normal \hat{n}ᵢ is only defined up to a rotation in the tangent plane. To work around this problem at a low computational cost, the approach described in Gainza 2020 is followed. Specifically, the first tangent vector ûᵢ=û(xᵢ) is oriented along the geometric gradient ∇^{û,\hat{v}}P(xᵢ) of a trainable potential P(xᵢ)=Pi=MLP(fᵢ), computed from the input features using a small MLP. Its gradient is approximated using a derivative of Gaussian filter on the tangent plane, implemented as a quasi-geodesic convolution:

$$\text{?}$$

$$\text{?} \text{indicates text missing or illegible when filed}$$

and then update the tangent basis (ûᵢ, \hat{v}ᵢ) using standard trigonometric formulae.

Local curvatures are computed in a similar fashion. Quasi-geodesic convolutions are used with Gaussian windows of radii σ that range from 1 Å to 10 Å and quadratic filter functions to estimate the local covariances Cov_{σ,i}^{û,\hat{v}}(p,p) and Cov_{σ,i}^{û,\hat{v}}(p,q) of the point positions and normals as 2×2 matrices in the tangent plane (ûᵢ, \hat{v}ᵢ). With λ=0.1 Å a small regularization parameter, the 2×2 shape operator at point xᵢ and scale σ is then approximated as S_{σ,i}=(λ²Id_{2×2}+Cov_{σ,i}^{û,\hat{v}}(p,p))⁻¹Cov_{σ,i}^{û,\hat{v}}(p,q), which allows us to define the Gaussian K K_{σ,i}=det(S_{σ,i}) and mean H_{σ,i}=trace(S_{σ,i}) curvatures at scale σ.

### Trainable Convolutions

Finally, the main building block of the architecture is a quasi-geodesic convolution that relies on a trainable MLP to weigh features in a geodesic neighborhood of the local reference point xᵢ. A vector signal fᵢ∈R^{F} is turned into a vector signal fᵢ′∈ [symbol] ^{F} with:

$$\text{?}$$

$$\text{?} \text{indicates text missing or illegible when filed}$$

where MLP is a neural network with 3 input units, H=8 hidden units, ReLU non-linearity and F=16 outputs.

### 3.3. End-to-End Convolutional Architecture

### Overview

The operations introduced in the previous sections are chained together to create the fully differentiable pipeline for deep learning on protein surfaces as shown in FIG. 2. As a brief summary:
1. Surface points and normals are sampled as shown in FIGS. 3A-3F.
2. The normals \hat{n}ᵢ are used to compute mean and Gaussian curvatures at 5 scales σ ranging from 1 Å to 10 Å.
3. Chemical features on the protein surface are computed as described in Section 3.1. Atom types and inverse distances to surface points are passed through a small MLP with 6 hidden units, ReLU non-linearity and batch normalization. Contributions from the 16 nearest atoms to a surface point xᵢ are summed together, followed by a linear transformation to create a vector of 6 scalar features.
4. These chemical features are concatenated to the 5+5 mean and Gaussian curvatures to create a full feature vector of size 16.
5. A small MLP is applied on this vector to predict orientation scores Pᵢ for each surface point. The local coordinates (\hat{n}ᵢ, ûᵢ, \hat{v}ᵢ), are then oriented according to (6).
6. Successive trainable convolutions (7) are applied, MLPs and batch normalizations on the feature vectors fᵢ. The numbers of layers, the radii of the Gaussian windows and the number of units for the MLPs are task dependent and detailed in the Supplementary Material section below.
7. As a final step for site identification, an MLP is applied to the output of the convolutions to produce the final site/non-site binary output. For interaction prediction, dot products are computed between the feature vectors of both proteins to use them as interaction scores between pairs of points.

### Asymmetry Between Binding Partners

When trying to predict binding interactions for protein pairs, both interacting proteins are processed identically up to the convolutional step. Some asymmetry is then introduced by passing each one of the two binding partners through a separate convolutional network. This allows the network to find complementary (instead of similar) regions on both surfaces, such as convex bulges and concave pockets. We note that MaSIF encoded such an asymmetry by inverting the sign of the precomputed features on one of the two surfaces.

### 4. EXPERIMENTAL EVALUATION

### Benchmarks

The method was tested on two tasks introduced in Gainza 2020. The tasks come from the field of structural bioinformatics and deal with predicting how proteins interact with each other.

Binding site identification: classifying the surface of a given protein into interaction sites and non-interaction sites is tried. Interaction sites are surface patches that are more likely to mediate interactions with other proteins: understanding their properties is a key problem for drug design and the study of protein interaction networks. The identification of the interaction site is unaware of the binding partner.

Interaction prediction: two surface patches are taken as inputs, one from each protein involved in a complex, and predict if these locations are likely to come into close contact in the protein complex. This task is key to prediction tasks like protein docking, i.e. predicting the orientation of two proteins in a complex.

### Dataset

The dataset comprises protein complexes gathered from the Protein Data Bank (PDB). The training/testing split described in Gainza 2020 is used. This training/testing split is based on sequence and structural similarity and was assembled to minimize the similarity between structures of the interfaces in the training and testing set. For site identification, the training and test sets include 2958 and 356 proteins, respectively; 10% of the training set is reserved for validation. For interaction prediction, the training and test sets include 4614 and 912 protein complexes, respectively, with 10% of the training set used for validation.

The average number of points used to represent a protein surface is N=11549±1853 for the generated point clouds, compared to 6321±1028 points for MaSIF. This smaller sampling size of MaSIF stems from the large time and memory requirements of this method, which prohibits the use of finer meshes. Proteins are randomly rotated and centered to ensure that methods which rely on atomic point coordinates do not overfit on their spatial locations.

### Baselines

The main baselines for evaluation are the MaSIF-site and MaSIF-search models described in Gainza 2020 and for the MaSIF baselines, the pre-trained models, precomputed surface meshes, and input features provided therein are used. Additionally, in order to show the benefits of the convolutional layer, it is benchmarked against PointNet++ and Dynamic Graph CNN (DGCNN), two popular state-of-the-art convolutional layers for point clouds.

### Implementation

The architectures were implemented with PyTorch and use KeOps for fast geometric computations. For data processing and batching, PyTorch Geometric was used. For the PointNet++ and DGCNN baselines, PyTorch Geometric implementations were used—but KeOps symbolic matrices are relied on to accelerate the construction of kNN graphs and thus guarantee a fair comparison. For the MaSIF baselines, the reference implementation described in Gainza 2020 was used. Since MaSIF is implemented in TensorFlow small discrepancies in measurements of memory consumption and running times are possible. All models were trained on either a single NVIDIA Geforce RTX 2080 Ti GPU or a single Tesla V100. Run times and memory consumption are measured on a single Tesla V100.

### 4.1. Surface and Input Feature Generation

### Precomputation

A key drawback of MaSIF is its reliance on the heavy precomputation of surface meshes and input features. These computations take a significant amount of time and generate large files that must be stored on disk. For reference, the pre-processed files used to train the MaSIF networks weigh more than 1 TB. In sharp contrast, the methods described herein do not rely on any such precomputation. Table 1 compares corresponding run times for both pipelines: the method described herein is three orders of magnitude faster than MaSIF for these geometric computations.

TABLE 1
Average “pre-processing” time per protein. The
dMaSIF method is about 1000 times faster than MaSIF
and allows these computations to be performed on the
fly, as opposed to the offline precomputations of MaSIF.

| | | |
|---|---|---|
| Computation | MaSIF | dMaSIF |

| | | | | |
|---|---|---|---|---|
| Surface generation | 6.11 ± 6.18 | s | 59.0 ± 15.2 | ms* |
| Input features | 19.69 ± 16.08 | s | 6.59 ± 1.22 | ms* |
| Local coordinates | 50.65 ± 45.15 | s | 0.46 ± 0.09 | ms* |

*With batches of 128 proteins at a time.

### Scalability

The dMaSIF surface generation algorithm scales beneficially with an increasing batch size. The supplemental material section below shows that the running time and memory requirement per protein of the dMaSIF method both decrease significantly when processing dozens of proteins at time the batch size. This is a consequence of the increased usage of the GPU cores and the smaller influence of fixed PyTorch and KeOps overheads.

Moreover, the dMaSIF method of surface generation makes it easy to experiment with different point cloud resolutions. Different tasks could benefit from higher or lower resolution and tuning it as a hyperparameter could have significant effects on performance. The effects of resolution on time an memory requirements are shown in more detail in the supplementary materials section below.

### Quality of Learned Chemical Features

Another notable drawback of MaSIF is its reliance on ‘handcrafted’ geometric and chemical features (Poisson-Boltzmann electrostatic potential, hydrogen bond potential and hydropathy) that must be precomputed and provided as input to the neural network. In contrast, the dMaSIF method does not use any handcrafted descriptors and instead learns problem-specific features directly from the underlying atomic point cloud, provided as the sole input of the method. This information alone is sufficient to compute an informative chemical and geometric description of the protein surface. In support of this FIG. 6A shows the results of an experiment where the chemical feature extractor of the dMaSIF method is used to regress the Poisson-Boltzmann electrostatic potential on surface points. In particular, FIG. 6A shows predicted Poisson-Boltzman electrostatic potential vs. the ground truth, which results in a Correlation cofactor r=0.83 and RMSE=0.16. The quality of the prediction suggests that the data-driven chemical features are of similar quality to the descriptors used by MaSIF—or better.

The results of an ablation study for chemical and geometric features are depicted in FIG. 6B. In particular, FIG. 6B shows how chemical and geometric features affect the performance in predicting interaction sites (ROC-AUC). They suggest that the concatenation of geometric curvatures to the vector of learned chemical features does not significantly improve the performance of the network for the site prediction task.

### 4.2. Performance

### Binding Site Identification

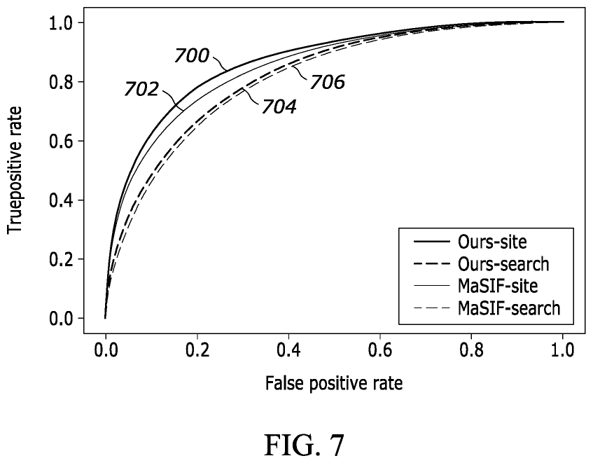

Results for the identification of binding sites are summarized in FIGS. 7-9, which depict ROC curves and tradeoffs between accuracy, time and memory. Multiple versions of the architecture were evaluated with varying numbers of convolution layers (1 vs 3) and patch sizes (5, 9, or 15A). For comparison, results are shown for when the convolutions are replaced by DGCNN and PointNet++ architectures, all other things being equal.

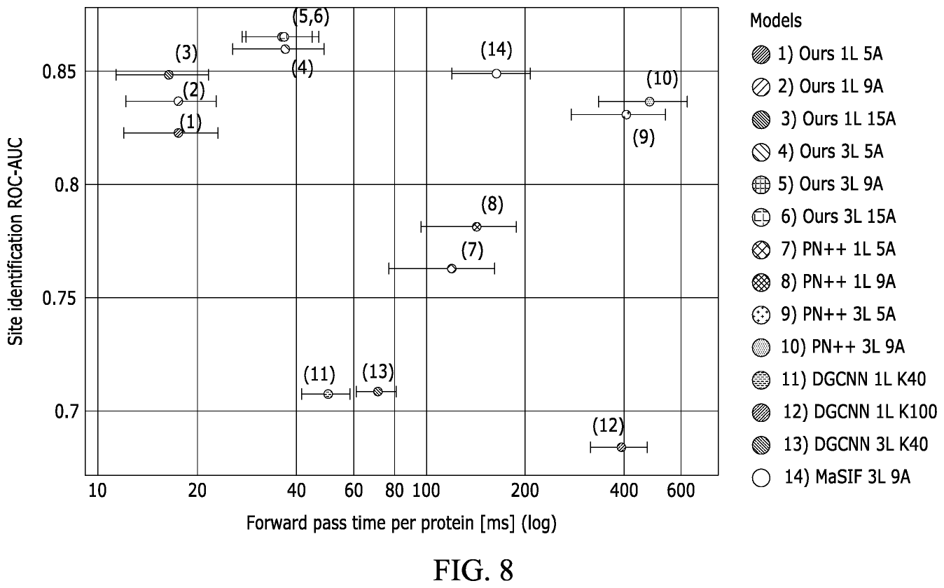

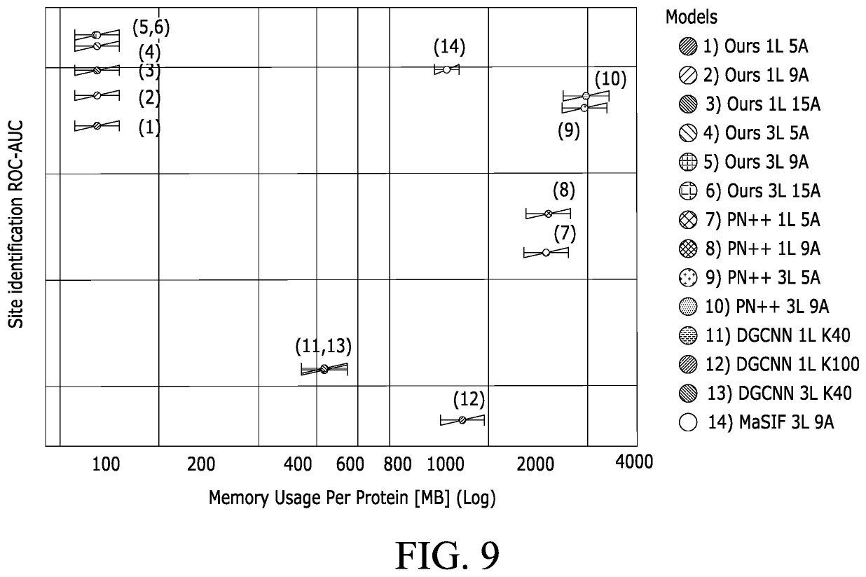

In particular, FIG. 7 shows ROC curves that compare performance of the dMaSIF method and MaSIF on the task of binding site identification (curves 700 and 702) and search of binding partners (curves 704 and 706). The dMaSIF method performs on par with MaSIF, achieving ROC-AUC of 0.87 (vs. 0.85) in site identification, and 0.82 (vs. 0.81) in identifying binding partners. FIG. 8 shows accuracy (site identification ROC-AUC) vs. run time (forward pass/protein in ms) of the different architectures. The models are identified by the convolutional operator used, number of convolutional layers, and the value of o used for the Gaussian window. PointNet++ models are identified by the radius of the neighborhood and DGCNN models by the number of nearest neighbors. FIG. 9 shows accuracy (site identification ROC-AUC) vs. memory footprint (MB/protein) of the different architectures.

A first remark is that where a single convolution layer with a Gaussian window of deviation a=15 A is used, the dMaSIF method matches the best accuracy of 0.85 ROC-AUC produced by MaSIF—with 3 successive convolutional layers on patches of radius 9 Å. In this configuration, the network runs 10 times faster than MaSIF with an average time in the forward pass of 16 ms vs. 164 ms per protein. At the price of a modest increase of the model complexity (three convolution layers, and 36 ms on average per protein), the dMaSIF method outperforms MaSIF with a 0.87 ROC-AUC, detailed in FIG. 7 (solid curves). Most remarkably, the models for the dMaSIF method all have a small memory footprint (132 MB/protein), which is 11 times less than an equivalent MaSIF network (1492 MB/protein), 13 times less than DGCNN (1,681 MB/protein) and 30 times less than PointNet++ (3,995 MB/protein).

### Interaction Prediction

With a single convolutional layer architecture similar to that of MaSIF-search the dMaSIF method reaches a slightly higher performance of 0.82 vs. 0.81, as illustrated in FIG. 7 (curves). MaSIF-search reaches this level of accuracy using high dimensional feature vectors with 80 dimensions compared to the 16 used in the dMaSIF method.

Note that MaSIF-search also relies on larger patches than MaSIF-site (12 Å vs. 9 Å), which causes a significant increase of run times to 727±403 ms. On the other hand, the lightweight dMaSIF method runs in 17.5±6.7 ms and is over 40 times faster at inference time.

### 5. CONCLUSION

The systems and methods described herein introduce a new geometric architecture for deep learning on protein surfaces, enabling the prediction of their interaction properties. This method is an order of magnitude faster and more memory efficient than previous approaches, making it suitable for the analysis of largescale datasets of protein structures: this opens the door to the analysis of entire protein-protein interaction networks in living organisms, comprising over 10K proteins.

The fact that the pipeline works on raw atomic coordinates and is fully differentiable makes it amenable to generative tasks, with the possibility of performing a true end-to-end design of new proteins for diverse biological functions, namely in terms of the design of binders for specific targets. This opens fascinating perspectives in drug design, including biologics for targeting disease relevant targets (e.g. cancer therapy, antiviral) that display flat interaction surfaces and are impossible to target with small molecules.

More broadly, the new algorithmic and architectural ideas for deep learning on 3D shapes through fast on-the-fly computations on point clouds, as described herein, are of general interest to computer vision and graphics experts.

### Supplementary Material

### 1. Description of Network Architectures

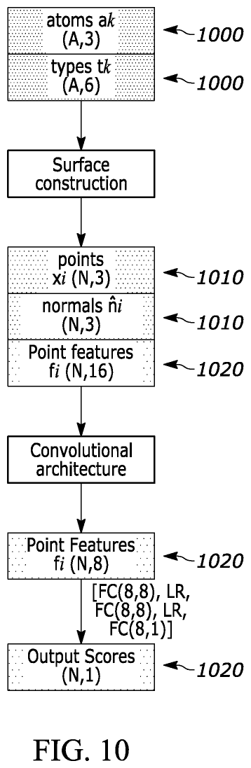

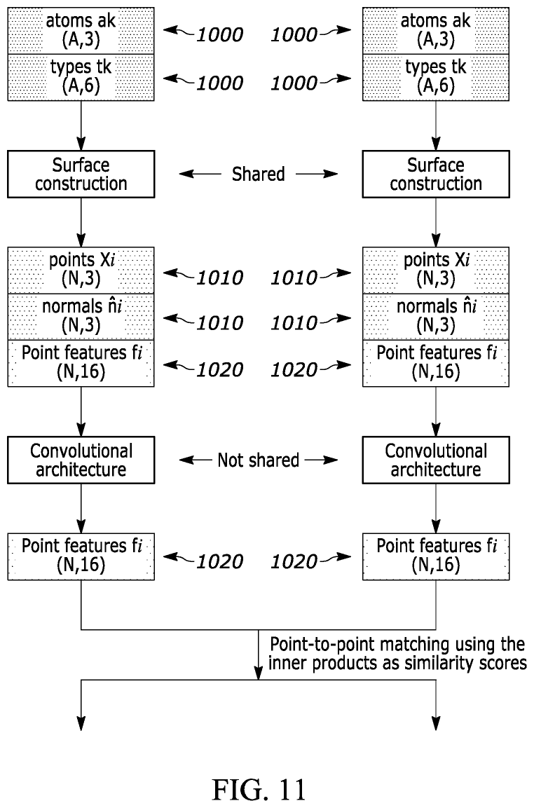

A high level description of the networks for both site identification and interaction prediction can be found in FIGS. 10 and 11 respectively. In these diagrams, “FC(I,O)” denotes a fully connected (linear) layer with I input channels and O output channels; “LR” denotes a Leaky ReLU activation function with a negative slope of 0.2; “BN” denotes a batch normalization layer. Blocks 1000, 1010, and 1020 in FIGS. 10-14 denote atom properties, surface descriptors and feature type vectors, respectively.

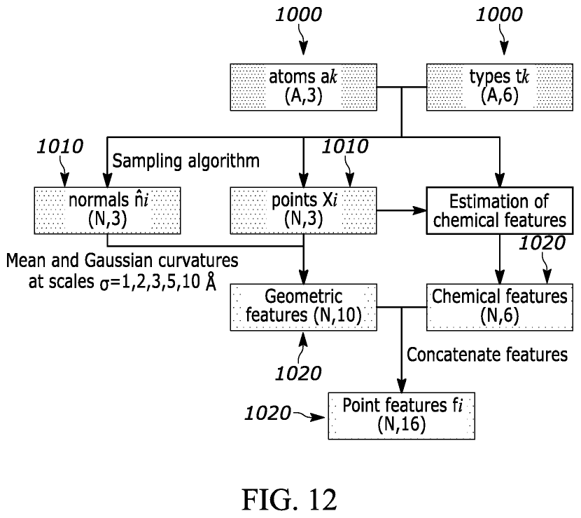

Furthermore, FIG. 10 shows an overview of the architecture for the site prediction task that is handled as a binary classification problem of the surface points. FIG. 11 shows an overview of the architecture for the search prediction task. FIG. 12 shows an overview of a “surface construction” block used for construction of a surface representation as detailed in Section 3.1 above.

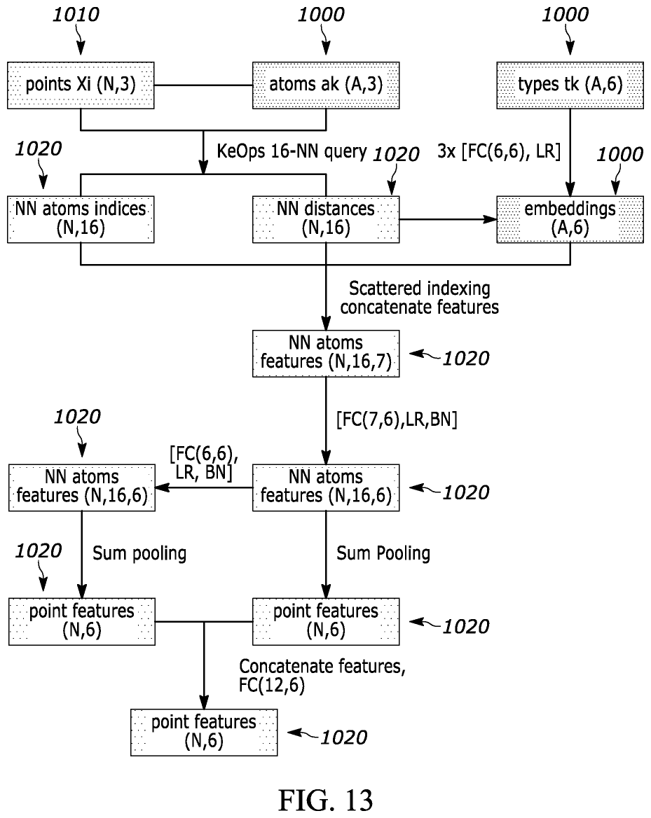

Chemical features are estimated on the generated surface points using the architecture shown in FIG. 13. In particular, FIG. 13 shows a “chemical features” block used for estimation of chemical features from the raw atom types and coordinates. This module takes as inputs the atom coordinates and types, along with the surface point coordinates. For each point on the surface, the network finds the 16 nearest atoms and assigns a 6-dimensional chemical feature based on the atom types and their distances to the point. As detailed in FIG. 12, these chemical features are concatenated to a 10-dimensional vector of geometrical features, which approximate the mean and Gaussian curvatures at different scales.

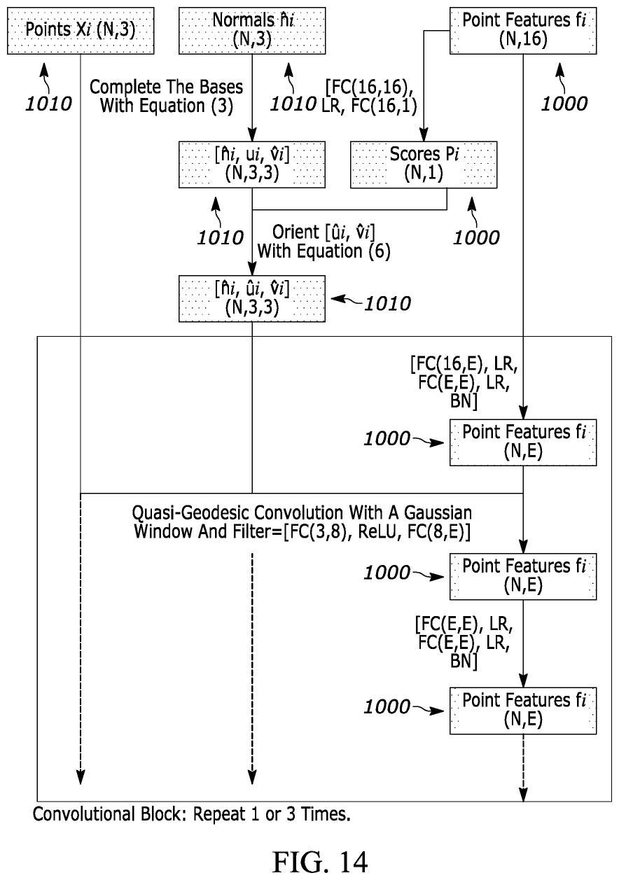

These input feature vectors are then passed through a sequence of convolutional layers as shown in FIG. 14. As discussed in Section 3, the surface normals \hat{n}ᵢ are first used to build local tangent coordinate systems and orient the unit tangent vectors ûᵢ, \hat{v}ᵢ according to the gradient of an orientation score Pᵢ. Finally, this complete description of the surface geometry is used to establish quasi-geodesic convolutional windows and progressively update our feature vectors.

In more detail, FIG. 14 shows the convolutional architecture, with E convolutional “channels” (E=8 is used for the site prediction task and E=16 for the search prediction task). The architecture for the search prediction task has an additional skip connection between the inputs and outputs. As detailed in Section 3.2, the network first estimates local coordinate systems [\hat{n}ᵢ, ûᵢ, \hat{v}ᵢ] attached to the points xᵢ of a protein surface. Then, a fast approximation of the geodesic distance is relied on to define quasi-geodesic convolutions and let feature vectors fᵢ interact on the protein surface.

The DGCNN and PointNet++ baselines replace the “convolutional” block of the architecture described herein with standard alternatives provided by PyTorch Geometric. The same numbers of channels are kept as for the method described herein (8 for the site prediction task, 16 for the search prediction task) and benchmark runs with several interaction radii and number of K-nearest neighbors.

### 2. Description of the Training Process

The datasets are filtered according to the criteria described in Gainza 2020. To be considered in the benchmarks, each protein must have at least 30 interface points and the interface has to cover less than 75% of the total surface area.

### Binding Site Identification

The hyperparameters are detailed in Table 2. Surfaces are generated in batches, but predictions are only performed on single proteins at a time. From each protein, 16 positives and 16 negatives, locations are randomly sampled and the loss function is computed on these points. This process was found to stabilize the training process and improve generalization. Labels are mapped from precomputed MaSIF meshes by finding the nearest neighbors. Furthermore, if a point is further than 2.0 Å away from any precomputed mesh point, it is labeled as non-interface. The loss is computed as the binary cross entropy between the labels and the predictions.

TABLE 1
Hyperparameters for our training loops.

| Parameter | Site | Search |
|---|---|---|
| Optimizer | AMSGrad | AMSGrad |
| Learning rate | 3 × 10⁻⁴ | 3 × 10⁻⁴ |
| Epochs | 50 | 100 |
| Descriptor dimensionality | 8 | 16 |
| Early stopping | Yes | Yes |

### Interaction Prediction

Surface generation and prediction are performed in the same way as for binding site identification. However, as detailed at the end of Section 3.3 above, each binding partner is passed through a separate convolutional network. The prediction scores are then computed by taking the inner product between the convolutional embeddings of the two proteins. Pairs of points are labeled as interacting if they are less than 1 Å from each other. From each protein, 16 positives and 16 negatives were randomly sampled. The loss was computed as the binary cross entropy.

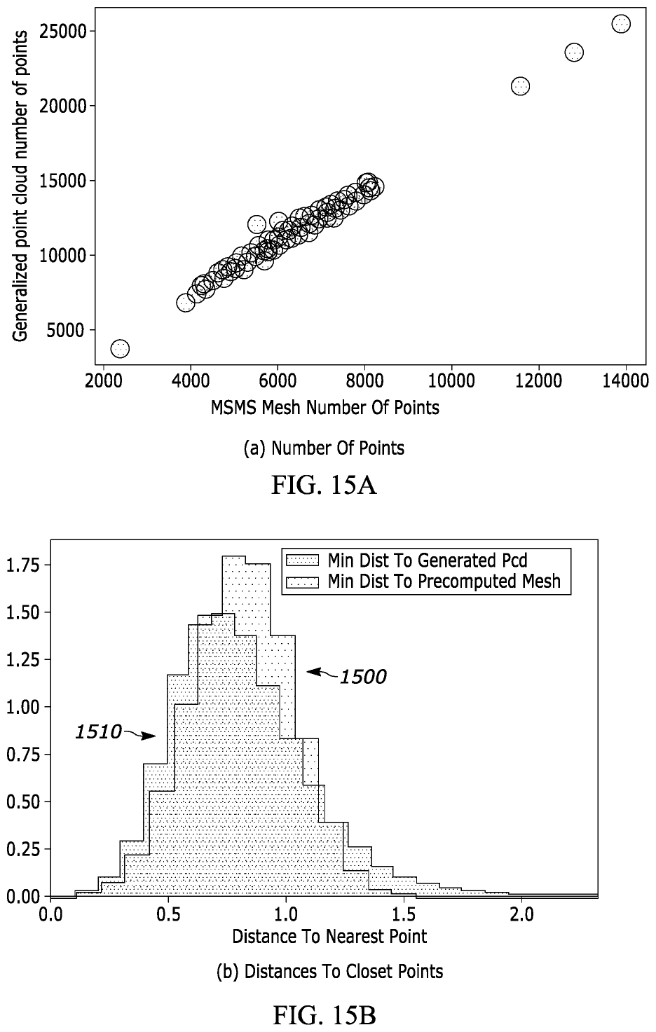

FIGS. 15A and 15B shows quality control results for the surface generation process of the dMaSIF method. In particular, FIG. 15A shows a number of points generated per protein by the dMaSIF method as a function of a number of points in the precomputed mesh used by the MaSIF method. As expected, a nearly perfect linear correlation is observed. FIG. 15B includes a plot 1500 showing a distance to the closest point on the precomputed mesh for each point generated by the dMaSIF method. FIG. 15B also shows, a histogram 1510 of distances to the closest generated point, for points on the MaSIF “ground truth” mesh. The histogram 1510 includes a very long tail (not visible in FIGB) that results from an artifact in the surface generation algorithm of MaSIF, which cuts out parts of proteins that have missing densities. This discrepancy is solved by removing these points from the dataset and only displaying point-to-point distances in the 99th percentile—i.e. the largest 1% distances are treated as outliers and not displayed in FIG. 15B.

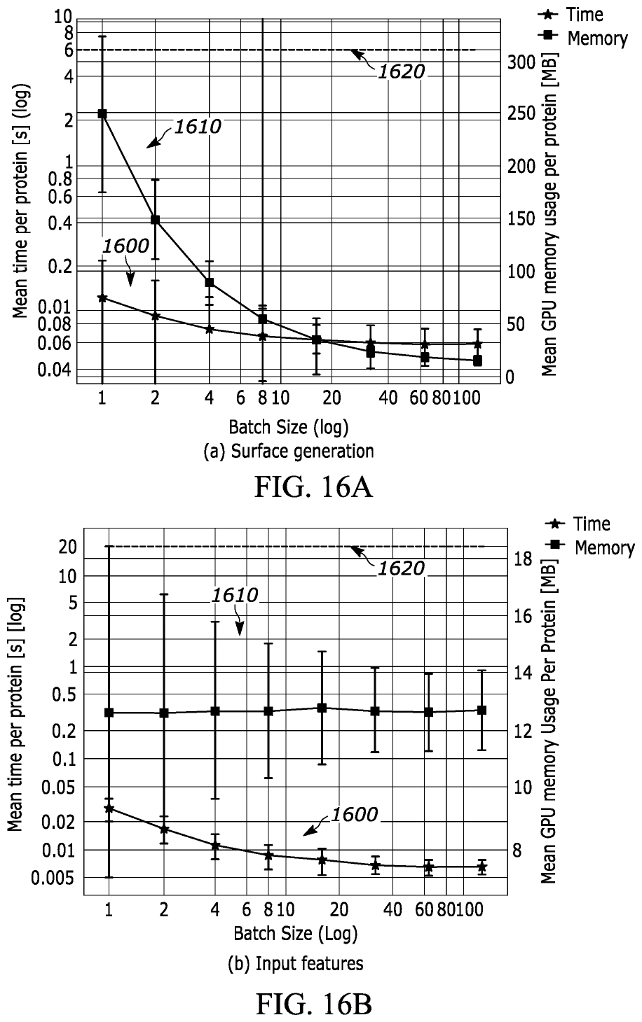

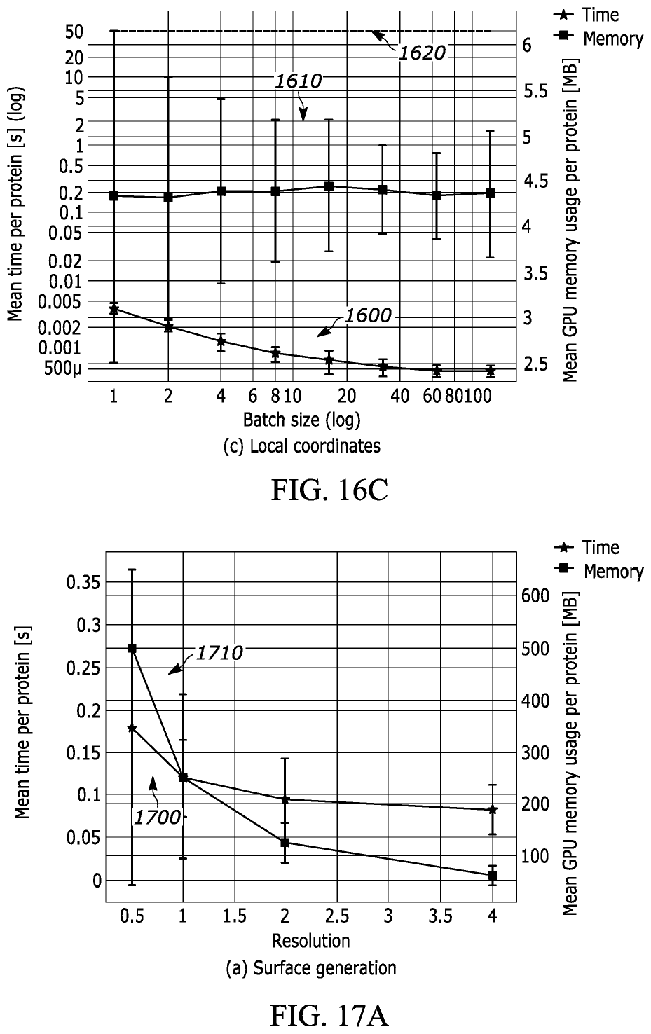

The graphs shown in FIGS. 16A-16C document a computational cost of the “pre-processing” routines of the dMaSIF method as functions of the batch size for the surface generation process (FIG. 16A), the input feature process (FIG. 16B), and the local coordinate process (FIG. 16C). The average time is shown by the curves 1600 and left axes in log scale and memory requirements are shown by the red curves 1610 and right axes in log scale. Both the time and memory requirements are shown per protein as a function of the number of proteins that are processed in parallel by the dMaSIF implementation. The dotted lines 1620 show the average time used by MaSIF to generate a surface mesh from the same atomic point cloud.

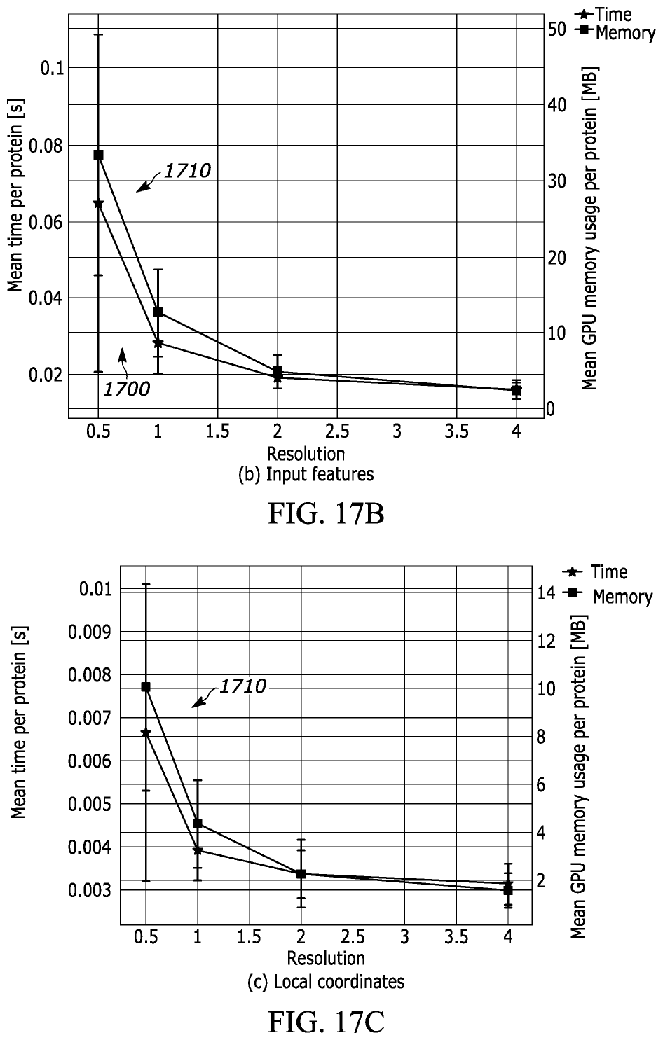

Similarly, the graphs shown in FIGS. 17A-16C document the computational cost of “pre-processing” routines of the dMaSIF method as a function of the sampling resolution for the surface generation process (FIG. 17A), the input feature process (FIG. 17B), and the local coordinate process (FIG. 17C). The time requirements (the lines 1700 and left axes) and the memory requirements (the lines 1710 and right axes) of the pre-convolutional steps of the dMaSIF architecture are shown in FIGS. 17A-16C as a function of the resolution of the generated point cloud. As expected, increasing the sampling density of the surface generation algorithm (i.e. using a lower resolution) results in longer processing times.

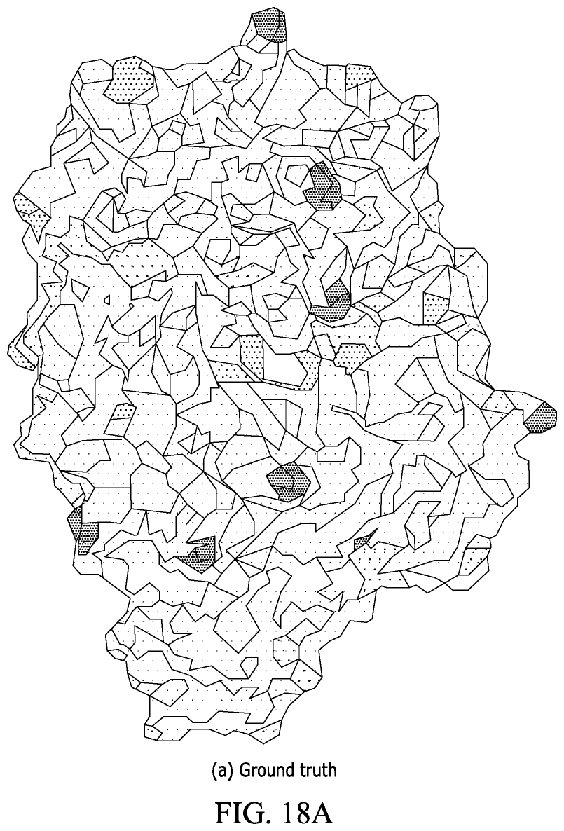

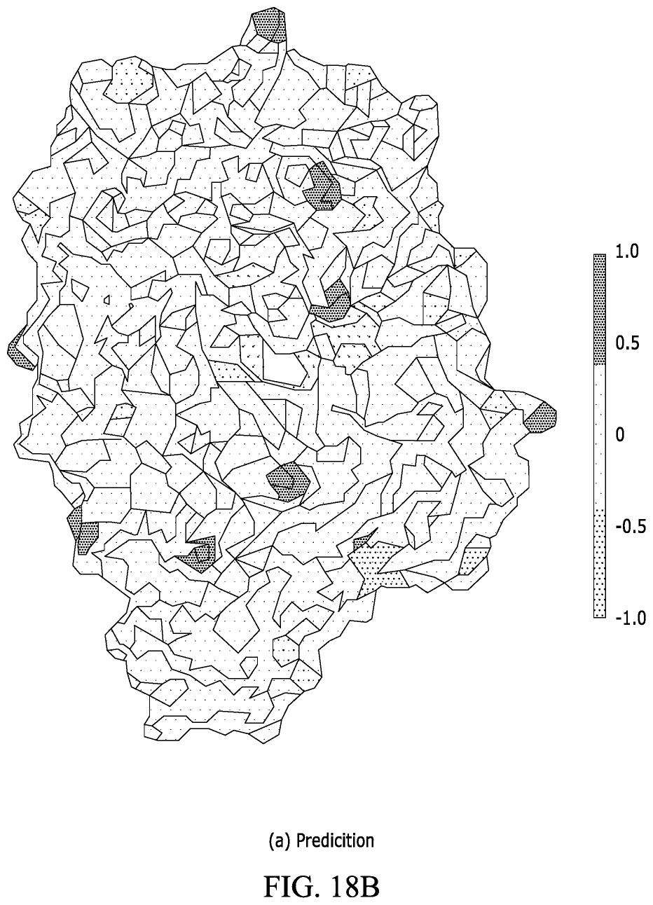

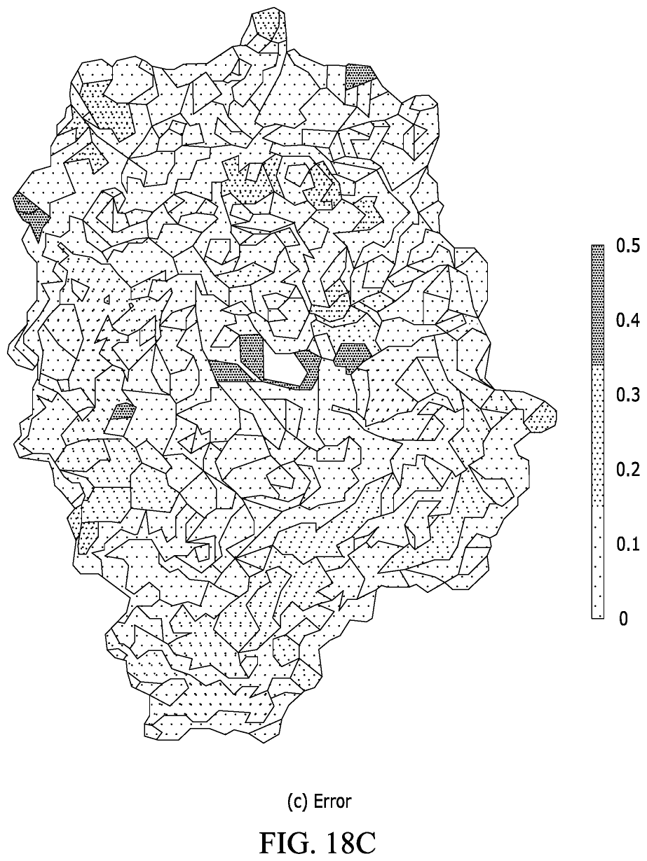

Additional renderings relating to the 10J7_D protein from the Protein Data Bank are shown in FIGS. 18A-18C. In particular, FIG. 18A shows the ground truth electrostatic potential on the protein surface, FIG. 18B shows the predicted electrostatic potential on the protein surface from the dMaSIF method, and FIG. 18C shows the error. The error is small, with RMSE=0.14. Furthermore, it is noted that most of the error is located inside the cavity.

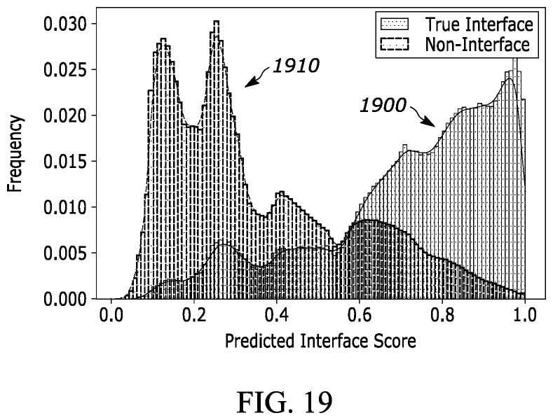

FIG. 19 shows distributions of predicted interface scores for both true interface points 1900 and non-interface points 1910 in relation to the site prediction task of the dMaSIF method. The separation is clear, resulting in a ROC-AUC of 0.87 as described in more detail above.

Further examples of the disclosed embodiments include:

1. A computer-system-implemented method for predicting properties of a protein molecule, comprising the steps of: receiving an input representation of the protein molecule; applying a surface generator to produce a molecular surface; applying at least one layer of geometric convolution on the molecular surface to produce a set of surface features; and using the set of features to predict the properties of the molecule.

5. A method according to example 1, wherein the input representation of the protein molecule is an atomic point cloud.

10. A method according to example 1, wherein the molecular surface is a point cloud.

12. A method according to example 10, wherein geometric convolution is performed on a point cloud molecular surface representation.

20. A method according to example 1, wherein the surface features are one or more of the follows: Geometric features; Curvature features; Electrostatic features; Hydropathy features; Poisson-Boltzmann features.

50. A method of example 1, wherein the steps of producing a molecular surface, applying at least one layer of geometric convolution, and predicting the properties are differentiable.

55. A method of example 1, wherein the step of producing a molecular surface is done on the fly.

60. A method of example 1, wherein the predicted properties of the molecule are its binding to another molecule.

70. A method of example 1, wherein the steps of producing a molecular surface, applying at least one layer of geometric convolution, and predicting the properties are parametric.

75. A method of example 70, wherein the parameters are determined by means of a training procedure.

100. A computer-system-implemented method for designing a protein molecule with desired properties, comprising the steps of: receiving a set of desired properties; producing an optimal input representation; applying a surface generator to produce a molecular surface; applying at least one layer of geometric convolution on the molecular surface to produce a set of surface features; and using the set of features to predict the similarity to the desired properties.

110. A method of example 1, wherein the step of producing an optimal input representation is obtained by means of an optimization procedure.

Further still, additional aspects of the disclosed embodiments can be understood with reference to the following clauses.

Clause 1. A computer-system-implemented method for predicting properties of a protein molecule, comprising the steps of: receiving an input representation of the protein molecule; applying a surface generator to produce a molecular surface; applying at least one layer of geometric convolution on the molecular surface to produce a set of surface features; and using the set of features to predict the properties of the molecule.

Clause 2. The method of clause 1, wherein the input representation of the protein molecule is an atomic point cloud.

Clause 3. The method of clause 1 or 2, wherein the molecular surface is a point cloud.

Clause 4. The method of any one of the preceding clauses, wherein the geometric convolution is performed on a point cloud molecular surface representation.

Clause 5. The method of any one of the preceding clauses, wherein the surface features are one or more of the following: geometric features; curvature features; electrostatic features; hydropathy features; Poisson-Boltzmann features.

Clause 6. The method of any one of the preceding clauses, wherein the steps of producing a molecular surface, applying at least one layer of geometric convolution, and predicting the properties are differentiable.

Clause 7. The method of any one of the preceding clauses, wherein the step of producing a molecular surface is done on the fly.

Clause 8. The method of any one of the preceding clauses, wherein the predicted properties of the molecule are its binding to another molecule.

Clause 9. The method of any one of the preceding clauses, wherein the steps of producing a molecular surface, applying at least one layer of geometric convolution, and predicting the properties are parametric.

Clause 10. The method of the preceding clause, wherein the parameters are determined by means of a training procedure.

Clause 11. A computer-system-implemented method for designing a protein molecule with desired properties, comprising the steps of: receiving a set of desired properties; producing an optimal input representation; applying a surface generator to produce a molecular surface; applying at least one layer of geometric convolution on the molecular surface to produce a set of surface features; using the set of features to predict the similarity to the desired properties.

Clause 12. The method of the preceding clause 11, wherein the step of producing an optimal input representation is obtained by means of an optimization procedure.

Clause 13. A data processing apparatus comprising means for carrying out the method of any one of clauses 1-12.

Clause 14. A computer program comprising instructions which, when the program is executed by a computer, cause the computer to carry out the method of any one of clauses 1-12.

1. A computer-system-implemented method for predicting properties of a protein molecule, comprising the steps of:
receiving an input representation of the protein molecule;
applying a surface generator to produce a molecular surface;
applying at least one layer of geometric convolution on the molecular surface to produce a set of surface features; and
using the set of surface features to predict the properties of the molecule.

2. The method of claim 1, wherein the input representation of the protein molecule is an atomic point cloud.

3. The method of claim 1, wherein the molecular surface is a point cloud.

4. The method of claim 1, wherein the geometric convolution is performed on a point cloud molecular surface representation.

5. The method of claim 1, wherein the set of surface features includes one or more of the following:
geometric features;
curvature features;
electrostatic features;
hydropathy features;
Poisson-Boltzmann features.

6. The method of claim 1, wherein the steps of producing the molecular surface, applying the at least one layer of geometric convolution, and predicting the properties are differentiable.

7. The method of claim 1, wherein the step of producing the molecular surface is done on the fly.

8. The method of claim 1, wherein the predicted properties of the molecule are binding of the molecule to another molecule.

9. The method of claim 1, wherein the steps of producing the molecular surface, applying the at least one layer of geometric convolution, and predicting the properties are parametric.

10. The method of claim 9, wherein the parametric steps are determined by a training procedure.

11. A computer-system-implemented method for designing a protein molecule with desired properties, comprising the steps of:
receiving a set of desired properties;
producing an optimal input representation;
applying a surface generator to produce a molecular surface;
applying at least one layer of geometric convolution on the molecular surface to produce a set of surface features;
using the set of surface features to predict similarity to the desired properties.

12. The method of claim 11, wherein the step of producing the optimal input representation is obtained via an optimization procedure.

13. (canceled)

14. A computer program comprising instructions which, when executed by a computer, cause the computer to carry out the method of claim 1.

15. A data processing apparatus for predicting properties of a protein molecule, the apparatus comprising:
one or more processors; and
one or memories having stored thereon computer-executable instructions that, when executed by the one or more processors, cause the computing system to:
receive an input representation of the protein molecule;
apply a surface generator to produce a molecular surface;
apply at least one layer of geometric convolution on the molecular surface to produce a set of surface features; and
use the set of surface features to predict the properties of the molecule.

16. The apparatus of claim 15, wherein the input representation of the protein molecule is an atomic point cloud.

17. The apparatus of claim 15, wherein the molecular surface is a point cloud.

18. The apparatus of claim 15, wherein the geometric convolution is performed on a point cloud molecular surface representation.

19. The apparatus of claim 15, wherein the set of surface features includes one or more of the following:
geometric features;
curvature features;
electrostatic features;
hydropathy features;
Poisson-Boltzmann features.

20. The apparatus of claim 15, wherein the molecular surface is produced on the fly.

21. The apparatus of claim 15, wherein the predicted properties of the molecule are binding of the molecule to another molecule.
